(19) United States
(12) Patent Application Publication
(10) Pub. No.: US 2024/0206896 A1
(43) Pub. Date: Jun. 27, 2024

(54) INTRAVASCULAR LITHOTRIPSY DEVICES AND SYSTEMS HAVING SPARK MONITORING FEEDBACK

(71) Applicant: Cardiovascular Systems, Inc., St. Paul, MN (US)

(72) Inventors: Gregory B. Ingersoll, Minneapolis, MN (US); Matthew D. Cambronne, North Oaks, MN (US); Joseph P. Higgins, Minnetonka, MN (US); Matthew W. Tilstra, Rogers, MN (US)

(21) Appl. No.: 18/393,260

(22) Filed: Dec. 21, 2023

Related U.S. Application Data
(60) Provisional application No. 63/434,912, filed on Dec. 22, 2022.

(51) Int. Cl. A61B 17/22; A61B 17/00; A61B 90/00

(52) U.S. Cl. CPC A61B 17/22022; A61B 90/08; A61B 2017/00292; A61B 2017/22025; A61B 2090/0807

(57) ABSTRACT

An intravascular lithotripsy catheter system for use in providing an energy wave a force to a thrombus or lesion within a vasculature, the catheter system including a catheter that extends from a proximal end to a distal end with a saline delivery lumen open from the distal end of the catheter for creating a controlled volume or bolus of saline at a distal region of the catheter, at least one pair of electrodes provided within the distal region of the controlled volume that are connectable with a high voltage pulse generator to create a spark across the electrode pair when inflated with a saline solution, and an optical fiber extending from the proximal end of the catheter distally to a position within the balloon for observing an optical phenomenon within the balloon when detectable light energy is generated by the optical phenomenon. Preferably, the optical fiber is connected with a photosensor at the proximal end of the catheter. Methods of using such a catheter system are also contemplated.

### CROSS-REFERENCE TO RELATED APPLICATIONS

This application claims priority to and the benefit of U.S. Provisional Application No. 63/434,912, filed Dec. 22, 2022, and entitled INTRAVASCULAR LITHOTRIPSY DEVICES AND SYSTEMS HAVING SPARK MONITORING FEEDBACK, the entire contents of which are incorporated herein by reference in their entireties.

### TECHNICAL FIELD

The present invention is directed to a catheter system for treating a vascular thrombus or calcified lesion or the like utilizing energy waves generated by electrodes within a conductive fluid medium.

### BACKGROUND

Catheter systems having an angioplasty balloon are commonly used to apply a physical force by expansion of the balloon against a calcified lesion within vasculature to force the calcification back into and against the blood vessel wall. Certain such calcified lesions and thrombi are not effectively broken up by the use of an angioplasty balloon.

More recently, catheter systems have been developed that include a balloon similar to an angioplasty balloon that is filled with a conductive liquid medium, such as a saline solution, for expanding the balloon in position at the lesion or thrombus, wherein the catheter system includes one or more pair(s) of electrodes operatively positioned within the conductive liquid medium. The electrodes are pulsed with high voltage direct current so as to create a spark that jumps over a gap between the two electrodes at each pulse. The spark within the conductive medium creates an energy wave that propagates through the liquid medium causing the balloon to physically provide a force against the lesion or thrombus. The energy propagation includes the creation of micro-bubbles that also facilitate the physical force. Such devices are known to provide an energy wave to act against a lesion or thrombus for purposes of breaking up the calcification or clotting.

Current catheter systems include a therapy sequence that includes a maximum number of continuous pulses, followed by a minimum delay time and a hard maximum total pulses associated with a particular catheter. One such product specifies:

| | 1 Hz (1 Pulse per | |
|---|---|---|
| Treatment Frequency | Second) | |
| Maximum Number of Continuous Pulses (1 | 30 | Pulses |
| cycle) | | |
| Minimum Pause Time | 10 | Seconds |
| Maximum Total Pulses Per Catheter | 300 | Pulses |

If a therapy is not yet completed after the maximum total pulse per catheter count, the physician must replace the catheter with the attendant undesirable, cost, delay, and distraction.

Intravascular lithotripsy (IVL) devices are available for some calcification patterns. Disposable IVL balloon devices are provided in different designs and sizes for peripheral or coronary indications. All designs utilize a reusable power source such as an IVL generator. One reusable DC generator comprises the following specification:

| | |
|---|---|
| Power | 110-240 VAC; 50-60 Hz; Single Phase, 15 A service |
| Size | 11″ (28.0 cm) high × 6″ (15.2 cm) wide × |
| | 11.5″ (29.2 cm) deep |
| Weight | 15 pounds (6.8 kg) |
| Output | Proprietary pulse delivery system. Output |
| | voltage 3000 volts peak, pulse frequency 1 Hz |
| Mobility | Product is designed to be mounted to an IV pole |
| Length | 5 ft (1.53 m) |
| Compatibility | Male key distally designed to connect only to catheter. |
| Operation | Lithotripsy pulsing is activated by pushing a |
| | button on the Connector Cable. |
| Use | Re-usable |

One such disposable device consists of a 0.014-inch guidewire-compatible, fluid-filled balloon angioplasty catheter with two lithotripsy emitters incorporated into the shaft of the 12-mm-long balloon segment. A fluid filled balloon (e.g. a 50/50 saline contrast medium) is inflated to about 4 atm and then electrical pulses are provided to the emitters that create high voltage sparks to provide the therapy. Acoustic waves are created and the calcium is fractured.

### SUMMARY

In one aspect, the present invention is directed to an intravascular lithotripsy catheter system for use in providing an energy wave a force to a thrombus or lesion within a vasculature, wherein the catheter system comprises a catheter that extends from a proximal end to a distal end with a saline delivery lumen open from the distal end of the catheter for creating a controlled volume or bolus of saline at a distal region of the catheter, at least one pair of electrodes provided within the distal region of the controlled volume that are connectable with a high voltage pulse generator to create a spark across the electrode pair when inflated with a saline solution, and an optical fiber extending from the proximal end of the catheter distally to a position within the balloon for observing an optical phenomenon within the balloon when detectable light energy is generated by the optical phenomenon. Preferably, the optical fiber is connected with a photosensor at the proximal end of the catheter.

The catheter system can further comprise a balloon near a distal end of the catheter that is operatively connected with the saline delivery lumen and that can be expanded by delivery of the controlled volume of saline. Preferably, the photosensor comprises a photodiode. In one embodiment, the optical fiber terminates within the controlled volume of saline with a distal tip that is directed toward a gap of the electrode pair.

The optical fiber preferably includes a cladding layer and in one embodiment, the distal end can be polished to receive the detectable light from the optical phenomenon. The provision of multiple optical fibers is contemplated and each optical fiber can be connected with an optical sensor. Moreover, plural electrode pairs is also contemplated wherein the multiple optical fibers can be directed to the plural electrode pairs.

In another embodiment, the optical fiber can be modified along its length to have plural fiber side portions that can receive light energy at plural locations along its length to be transmitted back to one or more photosensors. Such an arrangement can be done with plural electrode pairs or not. Such an arrangement can further comprise a Bragg grating more distal than the plural fiber side portions for reflecting light energy at a select wavelength proximally to the one or more photosensors.

In another aspect of the present invention, a method of using an intravascular lithotripsy catheter system can be used for providing an energy wave and a force to a thrombus or lesion within a vasculature, wherein the catheter system comprising a catheter that extends from a proximal end to a distal end with a saline delivery lumen open from the distal end of the catheter, at least one pair of electrodes provided within the distal region that are connectable with a high voltage pulse generator to create a spark across the electrode pair when inflated with a saline solution, and an optical fiber extending from the proximal end of the catheter distally for observing an optical phenomenon when detectable light is generated by the optical phenomenon, the optical fiber connected with a photosensor at the proximal end of the catheter, the method including creating a controlled volume or bolus of saline at a distal region of the catheter by delivering saline through the saline delivery lumen, generating a spark and thus a optical phenomenon across the electrode pair as located within of the controlled volume of saline, and detecting detectable light from the optical phenomenon by way of the optical fiber as also positioned within the controlled volume of saline at a photosensor at a proximal end of the optical fiber.

Such a method can include expanding a balloon by delivering the controlled volume of saline with the electrode pair and optical fiber also located within the balloon. The photosensor can comprise a photodiode that detects light at a select wavelength.

Within such method, the detecting step can be conducted by way of an end of the optical fiber that terminates within the controlled volume of saline with a distal tip that is directed toward a gap of the electrode pair. The optical fiber can includes a cladding layer and the distal end is polished for receiving the detectable light from the optical phenomenon. Alternatively, the detecting step can be conducted by way of the optical fiber that is modified along its length to have plural fiber side portions that can receive light energy at plural locations along its length to be transmitted back to one or more photosensors. In such a method, a Bragg grating can be positioned more distal than the plural fiber side portions, and the method includes reflecting light energy at a select wavelength proximally to the one or more photosensors. In the case of having plural electrode pairs, the method can include receiving light energy from the plural electrode pairs at the plural fiber side portions. In another alternative, plural electrode pairs can be provided and the method can comprises receiving light energy from the plural electrode pairs by way of multiple optical fibers that are directed to the plural electrode pairs.

A method of the present invention can detect an optical phenomenon that comprises a light leader emanating from an electrode of the electrode pair prior to the generation of a spark. Alternatively, a method of the present invention can detect an optical phenomenon that comprises a collapse of one or more microbubbles within the controlled volume of saline as such collapse of microbubbles creates light emanating as a result of sonoluminescence.

### BRIEF DESCRIPTION OF THE DRAWINGS

FIG. 1 illustrates a system for providing intravascular lithotripsy according to an aspect of the present invention.

FIG. 2 illustrates an inflated balloon in a vessel for providing intravascular lithotripsy according to an aspect the present invention.

FIG. 3 schematically illustrates an embodiment of the present invention with an optical fiber routed within an IVL balloon and for sensing generated light at a gap between an electrode pair.

FIG. 4 schematically illustrates another embodiment of the present invention for sensing generated light at multiple locations within an IVL balloon.

FIG. 5 schematically illustrates yet another embodiment of the present invention including the provision of an optically reflective structure distally of a sensor portion of an optical fiber.

FIG. 6 is an illustration of features of certain IVL catheters showing details of a working length of such an IVL catheter, an energy profile of the IVL catheter, and the provision of an optimized overlap zone within such an IVL catheter design.

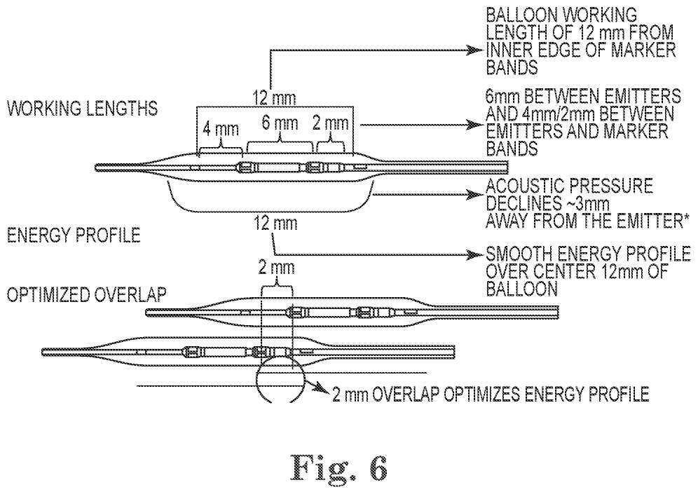

### DETAILED DESCRIPTION

The present invention is directed to IVL devices of the type that includes electrodes or lithotripsy emitters that create acoustic waves by arcing discharges between electrode components but may also include devices that create acoustic energy within the balloon via laser energy sources. Examples of such laser systems are described in U.S. Pat. Nos. 11,058,492 and 11,717,139 (the entire contents of which are incorporated by reference). Examples of electrically induced systems are described in U.S. Pat. Nos. 8,728,091, 9,642,673 and 10,850,078 and Published U.S. Pat. Appl. No. 2022-0054194 (the entire contents of which are incorporated by reference).

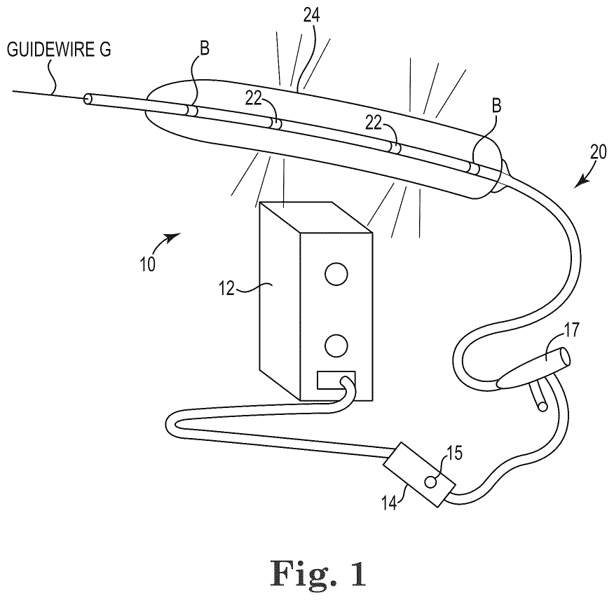

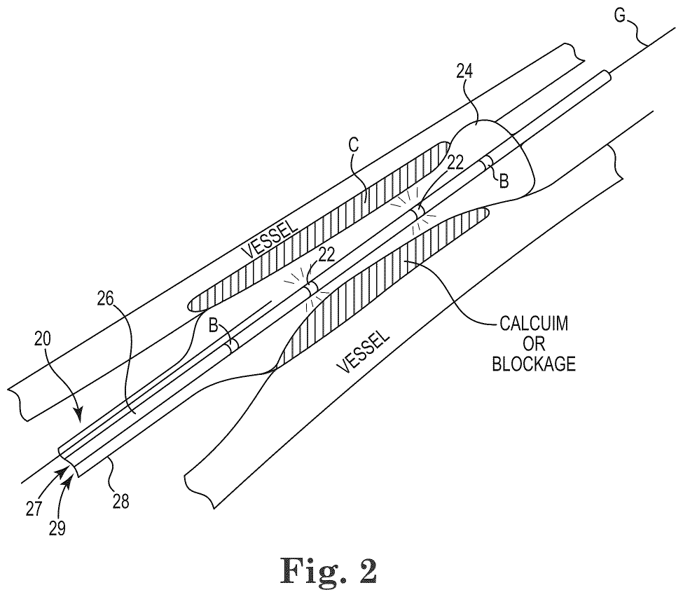

With reference to the Figures, FIGS. 1 and 2 show a system 10 according to the present invention comprising a power source 12 (in the form of an electrical generator, but alternatively in the form of a laser system), a handle 14 with therapy delivery control 15 and a catheter 20 with two lithotripsy emitters 22 (shown in the form of a pair of arcing electrodes, but alternatively they could comprise optical or laser emitters), and a fluid filled balloon 24. Optional marker bands B may be provided. The catheter 20 preferably includes a central tube 26 defining a guide wire lumen 27 through which a guide wire G passes for delivering the balloon 24 at the desired location along the guide wire G. A sheath 28 surrounds the central tube 26 and defines a delivery lumen 29 through which saline can be controllably delivered for balloon 24 inflation. The lumen 29 provides a concentric space around the central tube 26 within which electrode wires (not shown) can be run from the control 15 to the emitters 22 among other components in accordance with the present invention and discussed below. The sheath 28 is connected at a proximal end to a hub 17 that can include any number of ports allowing electrode wires to pass into the lumen 27 or 29 along with saline for inflation, the guide wire G, and any number of other components as desired.

The balloon 24 may be placed in a deflated position so as to more readily pass through a patient's vasculature to arrive at the scene of calcification. In use, the balloon 24 will be inflated to a common pressure for angioplasty procedures (e.g. 4 atm) and the therapy actuated via the delivery control 15.

FIG. 2 shows the balloon 24 inflated to a therapy delivery state where the lithotripsy emitters 22 may be “fired” to disrupt the vessel calcification C. Optional indicator bands B may be provided to afford visualization and proper positioning by use of known imaging techniques. The balloon 24 is inflated to a typical angioplasty pressure (e.g. 4 atm) and therapy is delivered. The balloon 24 may naturally expand during or just after the therapy is delivered to clear the vessel for passage of blood.

The control 15 is used to produce one or a series of voltage pulses in accordance with a treatment scheme. A high voltage pulse is provided to one of the emitters 22 comprising a pair of spaced electrodes and, in accordance with the illustrated embodiment, then in series to a second emitter 22 also comprising a pair of spaced electrodes. The high voltage pulse causes a spark across the first electrode pair then across the second electrode pair sequentially within the balloon 24. The somewhat conductive saline solution within the balloon 24 permits the high voltage spark across each electrode pair, thus creating an energy wave that propagates within the balloon toward the vessel calcification.

Any spark created within a balloon 24 that is located within a patient's vasculature will also create a visible, or detectable, light event. Such light event can be detected at wavelengths other than that of visible light. Moreover, it is understood that a visible or detectable light leader can emanate from the ground connected electrode of any electrode pair when a high voltage pulse is initiated on the hot electrode pair prior to the actual spark event. Such a leader is similar to that seen to occur from conductive objects prior to a lightning strike. It is an object of the present invention to monitor these visible or detectable light events as feedback to the controlled generation of a high voltage spark. Understanding the timing of the creation of such a leader as well as the actual spark in one or more electrode pairs after generation of the high voltage pulse can lead to design variations of the electrode pairs as well as other components for the propagation of the energy waves within the balloon. It is a purpose of the present invention to better predict the timing of spark generation from when the high voltage pulse is sent by sensing any optical phenomenon that can predict when a spark will actually occur. The establishment of a timing delay of the leader to the high voltage pulse and visualization of the resultant spark could also allow the system to gage in the proper timing, current, and electrode gaps are performing sufficiently within treatment specifications.

It is also contemplated that a catheter can be utilized in accordance with the present invention that does not include a balloon. Such a catheter would preferably include a lumen 29 that delivers saline to a controlled volume including the one or more emitters 22. Such a controlled volume can be created by structure of the vasculature of a patient along with the catheter distal end in the area of the emitters. Saline can be provided to fill such a controlled volume or may flow within and from such controlled volume at a controlled flow rate, creating a bolus of controlled fluid environment. A partial balloon is also contemplated from which saline fluid flow can weep from an open distal end of a partial balloon. Such a partial or open balloon design can be useful with a forward-facing electrode system such as disclosed within pending U.S. provisional patent application No. 63/416,231 filed Oct. 14, 2022, the entire contents of which are incorporated herein by reference. In the case of a catheter 20 having a balloon 24, the controlled volume is provided within the volume of the balloon 24. Having a direct pulse to pulse feedback system for an uncontrolled environment could be key to verify the proper environment is set up (fluid bolus/flow) and allowed the proper dielectric breakdown of the fluid, and subsequent bubble formation and collapse.

As also shown in FIG. 2, an optical fiber 30 is preferably run within the lumen 29 to a desired point within the balloon 24 for the purpose of sensing the occurrence of any light event within the balloon 24, such as may include a light leader or a spark. The optical fiber can also detect a non-light event, such as when a spark is expected but not generated. Such occurrences are discussed in greater detail below. A proximal end of the optical fiber 30 is preferably optically connected with an optical detector that preferably provides an amplified signal of the sensed light. Optical fibers are well known as comprising an optical core within which light can propagate that is surrounded by a cladding layer. The cladding layer may further be surrounded by a protective layer. Light such as generated by a spark or precursor leader can travel along the optical core to be sensed by the optical detector, such as a photodiode. By directly detecting the spark or leader occurrence by the observed visible or detectable light (via the optical fiber), longevity of the IVL device can potentially be improved by varying the power usage for each spark and optimizing the spark by intensity thereof. It is contemplated that reducing the spark intensity and duration can benefit the longevity of the IVL device and can reduce deleterious effects of heat that is generated by the spark. Also, control aspects and responsiveness can be improved by understanding spark intensity and timing relative to the high voltage pulses.

In the event of an electrical current high voltage pulse, such as can be monitored in accordance with the present invention, without a subsequent light emitting event (either leader or full spark or arc), diagnosing factors such as current rise, full current loss/delivery from storage mechanism, and/or indication to the user could be utilized to diagnose the system prior to subsequent electrical discharges. In products in which multiple serial electrodes are being fired/activated by a single high voltage pulse generator, the emission from single electrodes vs. current could be monitored to verify the total discharge vs. emission is appropriately representing the full proper discharge and therapy delivery. The medium in a controlled fluid environment, either closed or open, would also indicate to the system diagnostic information relative to the wavelength of the light being generated during the different stages. (e.g. sodium arc (saline or saline/contrast) vs. Hydrogen arc (water) vs. an arc representative of generation in blood pre characterized).

An additional optional use of the optical fiber 30 and an optical sensor is the detection of microbubbles within the saline solution of the balloon 24 that might interfere with generation of a single shockwave generating bubble collapse. It is understood that micro-bubbles are created by the high voltage spark, resultant energy wave, and electrolysis of the fluid in the balloon. It is also understood that such micro-bubbles can collapse under the application of certain energy to the micro-bubbles as suspended within a solution. Sonoluminescence is a phenomenon where the collapse of such a micro-bubble can result in both the generation of detectable light and sound waves. In the subject situation, a first pulse can create an energy wave and generate micro-bubbles with the saline solution within the balloon 24. In addition to monitoring and reacting to the light emitted during the activation of a spark, the fluid in the controlled volume could also be actively monitored by delivering a controlled-wavelength light source from an optical fiber and then capturing the absorption/reflection of the medium in the controlled environment. If for example, the fluid included bubbles or microbubbles the absorption/reflection and or wavelength would be affected indicating a non-controlled environment requiring intervention.

A subsequent pulse can create another energy wave and micro-bubbles, but can also cause the collapse of micro-bubbles within the saline solution from the earlier pulse. Such an event can generate other detectable light that can be detected by the optical fiber 30 during activation and/or on the termination of the activation and prior to the next activation of the electrodes. Such light generated from micro-bubble collapse might be detectable at a different wavelength that the spark detectable light, for example. Optical filters can be utilized depending on the optical phenomenon that is detected with an understanding of light emitted thereby at a specific wavelength. For example, sodium ion excitation in the saline solution can result from light emission in the 589 nm wavelength (yellow-orange) light. Detection of light at this wavelength could indicate properties of the spark and allow for adjustments to the spark generation and/or fluid environment surrounding the electrodes (e.g. adjusting the proportion of saline in the fluid).

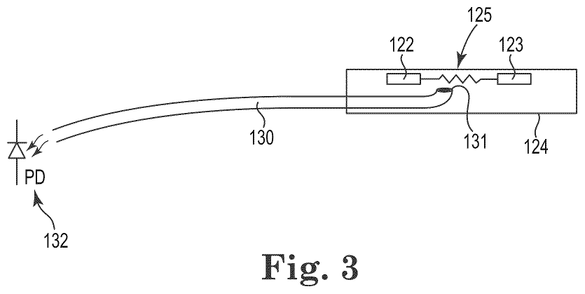

In one embodiment of the present invention an end of the optical fiber can be positioned nearby or adjacent to an electrode pair with a polished distal end of the optical fiber directed toward the electrode pair. FIG. 3 schematically illustrates such an arrangement, wherein an optical fiber 130 can be routed to a location within a balloon 124. Also within the balloon, a pair of electrodes 122 and 123 are arranged as above so as to define a gap 125 between the closest edges of the electrodes 122 and 123. Electrode 122 can be connected with the hot side of the high voltage pulse generator 12 by an electrical wire routed to the balloon 124 and the other electrode can be electrically connected by a wire also routed to the balloon 124 and connected with a ground side of the generator. By applying a high voltage pulse, as also described above, a spark can be created within the balloon 124 at the gap 125.

The optical fiber 130 preferably has a highly polished end 131 that is preferably located adjacent to the gap 125 and directed toward the gap 125 for sensing each generated spark and/or leader or any other detectable light event as a light occurrence. The zig-zag line between the electrodes 122 and 123 in FIG. 3 indicates a generated spark within the gap 125. A proximal end of the optical fiber can be connected with an optical sensor, such as a photodiode 132.

It is contemplated that multiple optical fibers can be routed to the electrode pair 122-123 for looking at the gap 125 from multiple different vantage points or to allow for pass-through monitoring of a light source and the subsequent interruption of that light. Such multiple vantage points can be from different distances or angles or both. For example, one may locate a distal end of an optical fiber as shown in FIG. 3 to sense and monitor the generated spark within the gap 125, while another distal end of an optical fiber may be directed at the ground electrode edge to sense and monitor that edge for the creation of a leader prior to the occurrence of the spark. Other variations are also contemplated for using multiple optical fibers at one spark gap for increasing the data associated with each spark and/or leader by, for example, directing light from the proximal end of each optical fiber to different optical sensors with filters for different light wavelengths.

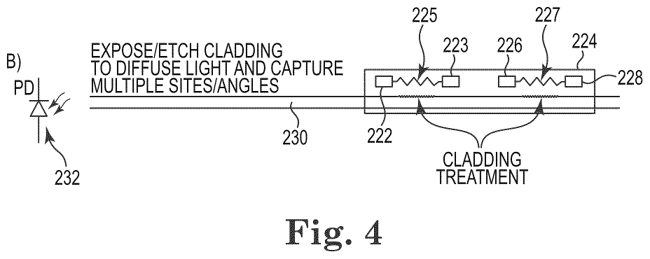

FIG. 4 illustrates the provision of a single optical fiber 230 that can sense and transmit light from multiple locations to an optical sensor such as a photodiode 232. Schematically illustrated are multiple electrode pairs connected in series and spaced from one another within balloon 224. A high voltage pulse will thus sequentially produce sparks from a first electrode pair including electrodes 222 and 223 and then from a second electrode pair including 226 and 228. These electrode pairs are electrically connected with the high voltage generator 12, as above to provide a hot electrode side 222, a ground side 223 and electrodes 226 and 228 connected as current travels in series over the sequential gaps 225 and 227 between the electrode pairs.

The optical fiber 230 is routed to the balloon and 224 and within the balloon 224 to pass nearby each gap 225 and 227. Instead of pointing or directing an end of an optical fiber to a gap, the optical fiber can be modified at each gap 225 and 227 (or more) so as to receive and transmit light back to the photodiode 232. Specifically, a portion of the cladding layer of the optical fiber can be removed at predetermined sites to correlate with the gaps 225 and 227. In this manner, it is a side of the optical fiber that collects light energy from multiple locations. The cladding layer and/or any protective layer can be etched or abraded or otherwise controllably removed at the predetermined locations to receive generated light within the core of the optical fiber. Such received light will propagate along the optical fiber to the photodiode 232, as above. The sequential sensing of light from the sequential sparks can be monitored in that manner.

It is contemplated that such a side sensing approach can also be utilized for a single gap monitoring. Similarly, multiple such fiber side modifications can be used to sense light at multiple locations from a single gap. As above, one sensor region of a fiber can look at a leader at a ground edge of an electrode 223 or 228 and another sensor region of the fiber can look at the gap and thus the spark. The optical fiber 230 can be routed so as to look at the same or different regions of the gap of the electrodes from different angles. Each of these aspects also apply to an optical fiber as routed near multiple gaps as well, as illustrated in FIG. 4. This side sensing approach can also be utilized to sense multiple locations, multiple electrodes, along a single fiber. This would reduce the bulk and complexity of delivering multiple fibers down a single catheter/system.

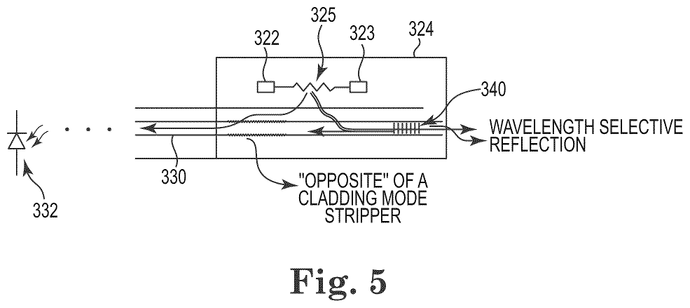

FIG. 5 schematically illustrates another aspect of the present invention when providing an optical fiber 330 with a side sensor region like described above and shown in FIG. 4. The optical fiber 330 can be routed past and nearby to a gap 325 formed between electrodes 322 and 323 and as provided within a balloon 324. In any arrangement with fiber side sensing, such as by removing a portion of the fiber cladding at one or more predetermined regions along the fiber side, light can enter the fiber core and transmit or propagate proximally to a photodiode 332 as an optical sensor. However, light will also transmit or propagate within the fiber core toward a distal end of the optical fiber 330. It is thus further contemplated to provide a reflector distal of the side sensor location(s) for intensifying the light that is transmitted to the photodiode 332.

Reflectors within optical fibers are well known and can be wavelength specific. A Bragg grating 340 can be provided within a short segment of the optical fiber 330 for reflecting light at a select wavelength. In FIG. 5, light is shown as transmitted both proximally and distally to a Bragg grating that reflects the lights back proximally. Light other than the select wavelength is transmitted distally. The desired wavelength is controlled by a periodic variation in the refractive index of the fiber core. The desired wavelength can then be matched with the photodiode 332 to better provide data with cleaner and stronger intensity. Plural such Bragg gratings 340 can be provided along a distal portion of the optical fiber 330 set to reflect different wavelengths of light. Other light filters can be provided proximally of the light sensing locations. In this regard, a light generating leader, for example, might have a greater intensity of light at one wavelength and the spark may have a greater intensity of light at another wavelength. Or, it may be desirable to analyze generated light at multiple wavelengths for different aspects of the generated light. Further it may be beneficial to reflect light at a wavelength associated with a leader to increase the signal to noise ratio at the detector while allowing some portion of high-intensity light associated with the spark to travel distally and be absorbed rather than reaching the detector possibly disrupting or damaging sensitive electronic components.

It is noted that the light discussed above from either a leader or spark includes visible light. However, detectable light other than visible light can be sensed and monitored at optical detectors based upon the desired light range to be monitored.

It is contemplated that multiple photodiodes or other photo sensors can receive light data transmitted from the light sensing at an optical fiber end or side regions, as described above. For example, multiple photodiodes can receive sensed light from one or more light splitters arranged at a proximal end of the optical fiber, which photodiodes can react cumulatively to the light data or can react to different aspects (wavelengths) of the transmitted light. Moreover, multiple optical fibers with side light sensing and/or fiber ends can be provided for redundancy. That is, spark events can potentially cause residue build up from sequential pulses such that inhibit the sensing of a good light signal. A redundant optical fiber can be positioned at a different vantage or angle relative to another. Such multiple optical fibers can be connected to a same photodetector or to plural such photodetectors. For example, if one optical fiber senses an optical phenomenon but the other does not, the other optical fiber could be damaged or covered with residue.

It is also contemplated that an optical fiber as above can be useful in detecting a spark leak or a pulse discharge to a patient. For example, if a high voltage pulse is generated and a spark is detected, but no current is sensed on the ground electrode of an electrode pair, the spark must be grounding elsewhere, such as to the patient. A current sensor anywhere along the ground wire side of the electrode pair would indicate a lack of ground current.

1. An intravascular lithotripsy catheter system for use in providing an energy wave and a force to a thrombus or lesion within a vasculature, the catheter system comprising:
a catheter that extends from a proximal end to a distal end with a saline delivery lumen open from the distal end of the catheter for creating a controlled volume or bolus of saline at a distal region of the catheter, at least one pair of electrodes provided within the distal region of the controlled volume that are connectable with a high voltage pulse generator to create a spark across the electrode pair when inflated with a saline solution, and
an optical fiber extending from the proximal end of the catheter distally to a position within the controlled volume of saline for observing an optical phenomenon within the controlled volume of saline when detectable light is generated by the optical phenomenon, the optical fiber connected with a photosensor at the proximal end of the catheter.

2. The catheter system of claim 1, further comprising a balloon near a distal end of the catheter that is operatively connected with the saline delivery lumen and that can be expanded by delivery of the controlled volume of saline.

3. The catheter system of claim 2, wherein the photosensor comprises a photodiode.

4. The catheter system of claim 1, wherein the optical fiber terminates within the controlled volume of saline with a distal tip that is directed toward a gap of the electrode pair.

5. The catheter system of claim 4, wherein the optical fiber includes a cladding layer and the distal end is polished to receive the detectable light from the optical phenomenon.

6. The catheter system of claim comprising multiple optical fibers, each optical fiber connected with an optical sensor.

7. The catheter system of claim 6, further comprising plural electrode pairs, and wherein the multiple optical fibers are directed to the plural electrode pairs.

8. The catheter system of claim 1, further comprising plural electrode pairs, wherein the optical fiber is modified along its length to have plural fiber side portions that can receive light energy at plural locations along its length to be transmitted back to one or more photosensors.

9. The catheter system of claim 8, further comprising a Bragg grating more distal than the plural fiber side portions for reflecting light energy at a select wavelength proximally to the one or more photosensors.

10. A method of using an intravascular lithotripsy catheter system for providing an energy wave and a force to a thrombus or lesion within a vasculature, the catheter system comprising a catheter that extends from a proximal end to a distal end with a saline delivery lumen open from the distal end of the catheter, at least one pair of electrodes provided within the distal region that are connectable with a high voltage pulse generator to create a spark across the electrode pair when inflated with a saline solution, and an optical fiber extending from the proximal end of the catheter distally for observing an optical phenomenon when detectable light is generated by the optical phenomenon, the optical fiber connected with a photosensor at the proximal end of the catheter, the method including:
creating a controlled volume or bolus of saline at a distal region of the catheter by delivering saline through the saline delivery lumen,
generating a spark and thus a optical phenomenon across the electrode pair as located within of the controlled volume of saline
detecting detectable light from the optical phenomenon by way of the optical fiber as also positioned within the controlled volume of saline at a photosensor at a proximal end of the optical fiber.

11. The method of claim 10, wherein the catheter further comprises a balloon near a distal end of the catheter that is operatively connected with the saline delivery lumen and the method includes expanding the balloon by delivering the controlled volume of saline.

12. The method of claim 10, wherein the photosensor comprises a photodiode that detects light at a select wavelength.

13. The method of claim 10, wherein the detecting step is conducted by way of an end of the optical fiber that terminates within the controlled volume of saline with a distal tip that is directed toward a gap of the electrode pair.

14. The method of claim 10, wherein the detecting step is conducted by way of the optical fiber that includes a cladding layer and the distal end is polished for receiving the detectable light from the optical phenomenon.

15. The method of claim 10, wherein the detecting step is conducted by way of the optical fiber that is modified along its length to have plural fiber side portions that can receive light energy at plural locations along its length to be transmitted back to one or more photosensors.

16. The method of claim 15, wherein the optical fiber further comprises a Bragg grating more distal than the plural fiber side portions, and the method includes reflecting light energy at a select wavelength proximally to the one or more photosensors.

17. The method of claim 16, wherein the catheter comprises plural electrode pairs and wherein the method comprises receiving light energy from the plural electrode pairs at the plural fiber side portions.

18. The method of claim 10, wherein the catheter comprises plural electrode pairs and wherein the method comprises receiving light energy from the plural electrode pairs by way of multiple optical fibers that are directed to the plural electrode pairs.

19. The method of claim 10, wherein the optical phenomenon comprises a light leader emanating from an electrode of the electrode pair prior to the generation of a spark.

20. The ;method of claim 10, wherein the optical phenomenon comprises a collapse of one or more microbubbles within the controlled volume of saline as such collapse of microbubbles creates light emanating as a result of sonoluminescence.
